(19) United States
(12) Patent Application Publication
(10) Pub. No.: US 2016/0136409 A1
(43) Pub. Date: May 19, 2016

(54) SUPPORT DEVICE FOR INTRAVENOUS LINE

(71) Applicant: Michael Mydlarz, Stamford, CT (US)

(72) Inventor: Michael Mydlarz, Stamford, CT (US)

(21) Appl. No.: 13/564,710

(22) Filed: Aug. 1, 2012

Related U.S. Application Data
(60) Provisional application No. 61/513,883, filed on Aug. 1, 2011.

(51) Int. Cl. A61M 39/00

(52) U.S. Cl. CPC A61M 39/00

(57) ABSTRACT

A support device for an IV line includes an IV line support mechanism, a suspension line structured for suspending the support mechanism on a portion of a patient's body, and a suspension line length adjustment mechanism for gripping and retaining portions of the suspension line so as to form an adjustable loop.

### CROSS REFERENCE TO RELATED APPLICATIONS

This application claims the benefit of U.S. Provisional Application Ser. No. 61/513,883 filed on Aug. 1, 2011.

### BACKGROUND OF THE INVENTION

The embodiments of the present invention relate to a device for supporting a portion of one or more intravenous (IV) lines connected to associated ports of one or more Hickman lines or other types of catheters.

Catheters may be surgically inserted into a vein through a patient's chest wall or at another location, and may be sutured to the patient's skin to prevent the insertion end from slipping out. These catheters can be in place for months or even years at a time. Typically, the external port end of the catheter is looped and secured to the skin with tape or is pinned to the patient's shirt, undergarment or bra. The catheter port may be attached to an intravenous (IV) line or drain line which is connected to an IV bag, drainage bag, or other device at an opposite end of the IV line.

Movement and exercise are made difficult and uncomfortable if the clothing that the catheter is pinned to moves in relation to the insertion point of the catheter. Sometimes, the catheter is accidentally pulled out because the patient forgets to detach it from their clothing when undressing, or because of forces acting on the IV line due to the weight of the IV line or movement of the IV bag with respect to the patient. Such problems occur even in acute catheter use for a brief time. In addition, the insertion site of the catheter is often the site of infections. Inadvertent extraction of the catheter from the insertion site may produce injury and promote such infections.

Thus, a need exists for a means of relieving the stresses exerted by an attached IV line on a catheter inserted into a patient's body.

### SUMMARY OF THE INVENTION

In one aspect of the embodiments of the present invention, a support device for an IV line includes an IV line support mechanism, a suspension line structured for suspending the support mechanism 12 on a portion of a patient's body, and a suspension line length adjustment mechanism for gripping and retaining portions of the suspension line so as to form an adjustable loop.

### BRIEF DESCRIPTION OF THE DRAWINGS

FIG. 1 is a view of a support device for an intravenous line in accordance with one embodiment of the present invention.

FIG. 2 is a view of an IV line support mechanism in accordance with one embodiment of the present invention.

FIG. 3 is a view of an embodiment of the support device in use by a patient to support an associated intravenous line.

FIG. 4A is a view of a closed state of a suspension line length adjustment mechanism in accordance with an embodiment of the present invention.

FIG. 4 is a view of an open state of the suspension line length adjustment mechanism embodiment shown in FIG. 4A.

FIG. 5 is a view showing a support device in accordance with one embodiment of the present invention supporting a triple lumen Hickman line.

FIG. 6A is a side view of a clip in accordance with an embodiment of the present invention for supporting a single IV line.

FIG. 6B is a front view of the clip shown in FIG. 6A.

FIG. 7A is a front view of a clip in accordance with another embodiment of the present invention.

FIG. 7B is a side view of the clip shown in FIG. 7A.

FIG. 7C is a side view of the clip of FIG. 7A engaging an IV line.

### DETAILED DESCRIPTION

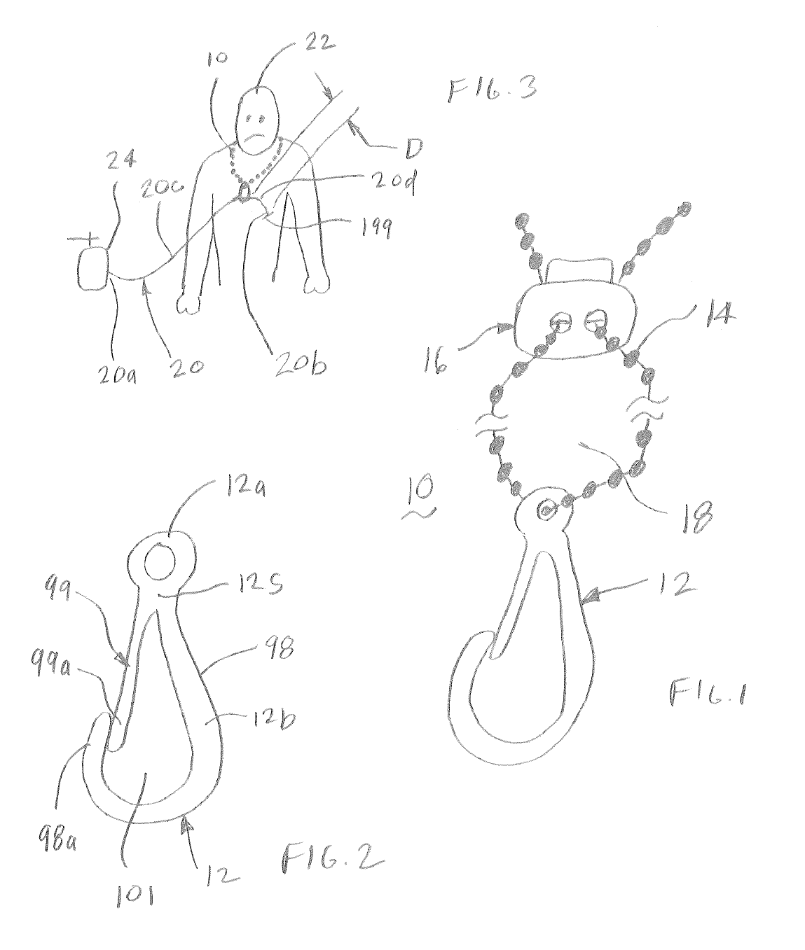

Referring to FIGS. 1-4B, a support device 10 for an IV line in accordance with one embodiment of the present invention includes an IV line support mechanism 12, a suspension line 14 configured for suspending the support mechanism 12 on a portion of a patient's body, and a suspension line length adjustment mechanism 16 to grip and retain portions of the suspension line 14 so as to form an adjustable loop 18.

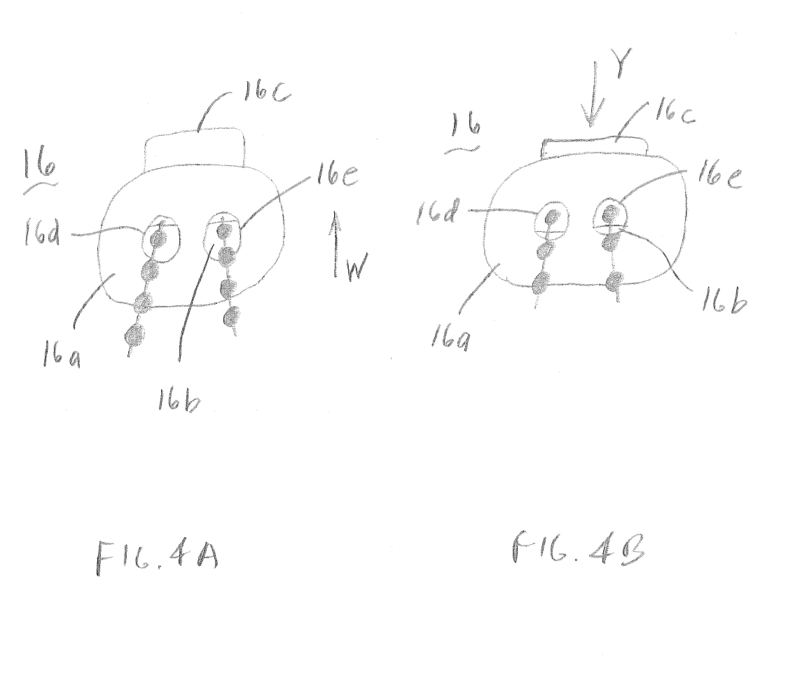

FIG. 2 shows an IV line support mechanism 12 in accordance with one embodiment of the present invention. In this embodiment, support mechanism 12 includes a suspension line receiving portion 12a for receiving a portion of a suspension line 14 (not shown) therethrough, and an IV line retaining portion 12b for securing the support mechanism 12 to an IV line 20 (as seen in FIG. 3) at a desired location along the line. However, the line support mechanism 12 may have any alternative configuration suitable for the purposes described herein. IV line support mechanism 12 may be formed from any suitable material. In a particular embodiment, the line support mechanism 12 is formed from a polymer material or other non-metallic material so that the line support mechanism 12 may be worn by a patient during a magnetic resonance imaging (MRI) scan.

In the embodiment shown in FIGS. 1 and 2, support mechanism retaining portion 12b includes a base portion 98 and a deflectable locking portion 99 coupled to the base portion to define a loop 101 sized for receiving a portion(s) of one or more IV lines therethrough.

As seen in the drawings, an end 99a of locking portion 99 overlaps an end 98a of base portion 98 within the interior of loop 101 and is arranged so as to be resiliently deflectable in a direction toward or into the loop 101. In addition, motion of the locking portion in a direction away from or outside the loop 101 is prevented by abutment of locking portion end 99a with the overlapping end 98a of base portion 98.

The locking portion 99 may be deflected inwardly toward the loop to create a gap between locking portion end 99a and base portion end 98a. The IV line(s) may then be inserted into the loop through this gap. Alternatively, the IV line(s) may be fed through the loop 101 without deflecting locking portion 99 to create the gap.

In a particular embodiment, an opening defined by suspension line receiving portion 12a is separated from the interior of loop 101 by a wall or separator 12s, as shown in FIG. 2. This prevents contact between the suspension line 14 and an IV line received within loop 101.

In a particular embodiment, the IV line retaining portion 12b is structured such that one or more ends of loop 101 narrow or taper, defining a relatively larger opening in one portion of the loop and tapering down to a relatively smaller opening size. This enables IV lines having any of a variety of diameters to be engaged by walls of the retaining portion, by moving the IV line along the loop 101 until it is engaged between opposed walls of the retaining portion. This also enables IV lines of a variety of different diameters to be received within loop 101, and enables the loop to receive multiple IV lines in at least a portion of the loop.

FIG. 5 is a view showing a support device in accordance with one embodiment of the present invention supporting a known triple lumen Hickman line 230. In the embodiment shown, one of the constituent lumens (in this case, lumen 232) is inserted into loop 101 of retaining portion 12b after the individual lumens 232, 234, and 236 contained in the main line 242 divide at junction 240. Loop 101 is sized to prevent junction 240 from passing through the loop. Thus, the suspension line 14 may be tightened or adjusted so that the retaining portion engages the junction 240, to support the weight of the junction 240 and the individual lumens extending from the junction. In this manner, pressure on the exit site incision exerted by the weight of the junction 240 and the individual lumens 232, 234, and 236 is relieved by the retaining portion 12b.

The loop 101 may be configured to receive therethrough any desired number of individual lumens in the manner described above, so long as the configuration of retaining portion 12b prevents junction 240 from passing through loop 101. In addition, any desired lumen extending from the main Hickman line 242 may be passed through loop 101 to support the junction and individual lines in the manner described above.

In one embodiment, the retaining portion 12b is structured to engage an IV line having a diameter within the range of 2 millimeters to 4 millimeters.

In these embodiments, the engagement between the retaining portion 12b and the IV line 20 prevents the line from being pulled through the loop 101 in either direction. Thus, the length D of IV line 20d between the retaining portion 12b and the incision in the patient into which the IV line extends remains the same regardless of the patient's movements or stresses exerted on line portion 20c.

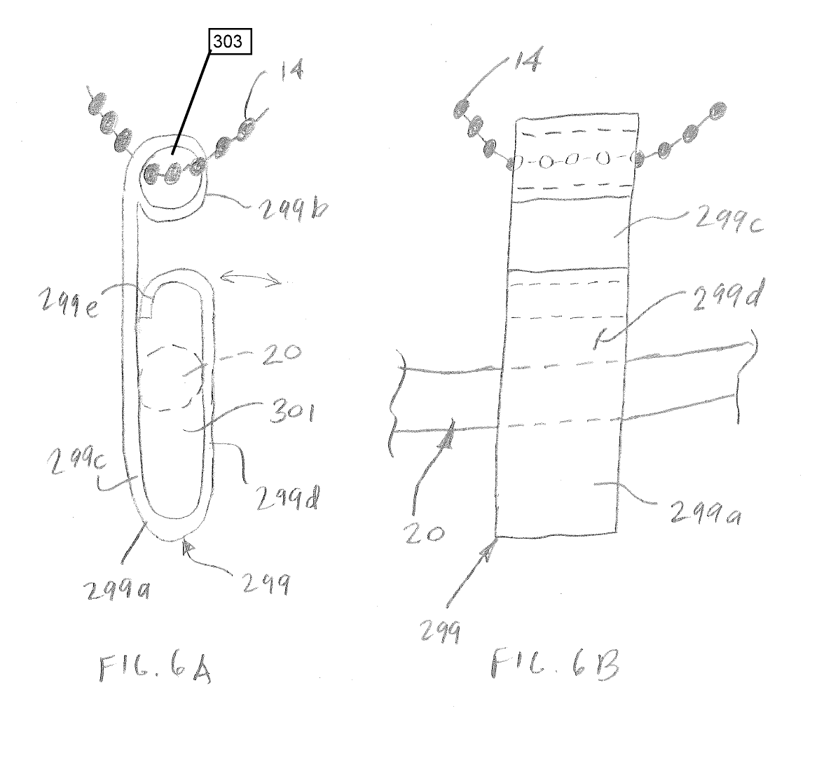

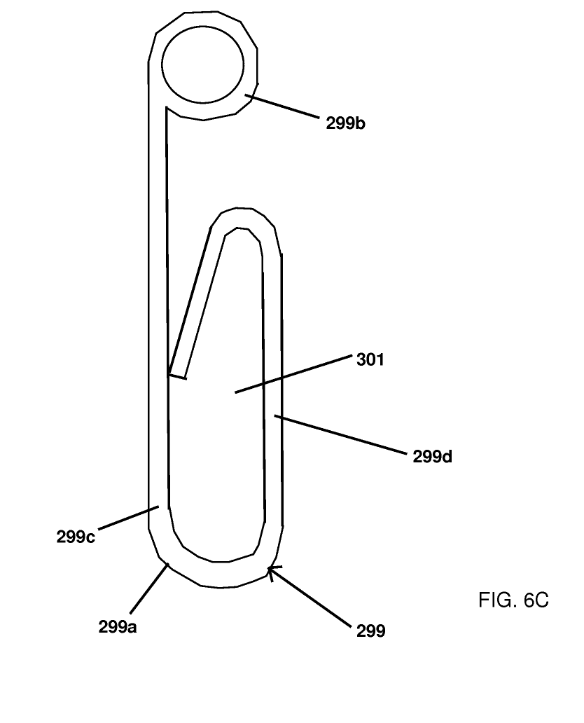

Referring to FIGS. 6A-6B, in a particular embodiment, a clip 299 is provided for engaging an IV line 20 in a slight interference fit, so as to hold and retain the engaged portion of the IV line while minimizing constriction of the IV line.

In the embodiment shown in FIGS. 6A and 6B, clip 299 has an IV line retaining portion 299a, and an attachment portion 299b including an opening 303 usable for attaching the clip 299 to suspension line 14 and/or support mechanism 12. Attachment portion 299b may be supported by support mechanism 12 by inserting the support mechanism base portion through opening 303 and suspending the clip 299 from the support mechanism. Alternatively, the clip may be directly attached to the suspension line 14 by passing the suspension line through opening 303.

In a particular embodiment, retaining portion 299a includes a base portion 299c and an arm 299d extending from the base portion opposite the base portion and structured to be resiliently deflectable with respect to the base portion. Base portion 299c and arm 299d combine to define a cavity or loop, generally designated 301, structured for receiving a portion of an IV line therein. Arm 299d may have a hooked end 299e or other feature shaped to prevent inadvertent extraction of an IV line from loop 301. In a particular embodiment (not shown), the hooked end of arm 299d is straight and angled with respect to base portion 299c so as to provide a tapered lead-in surface leading into loop 301.

Clip is structured so that the size of cavity 301 and the reaction forces exerted by arm 299d (when in a deflected state due to the presence of the IV tube) is sufficient to prevent or retard motion of the secured portion of the IV line out of the cavity, while minimizing constriction of the IV line.

The clip may be structured to secure any IV line having a diameter residing within a predetermined range of values. In one particular embodiment, the clip is structured so that the range of IV line diameters securable within cavity 301 in the manner described above is 2-5 millimeters.

To insert a portion of an IV line into cavity 301, arm 299d is deflected away from base portion 299c and the portion of the IV line inserted into the cavity. When positioned within cavity 301, arm 299d and base 299c engage the inserted portion of the IV line in an interference fit, as previously described.

In one embodiment, clip 299 is formed from a resiliently deflectable material (such as a polymer, for example). In a particular embodiment, clip 299 is formed from a polymer material, another non-metallic material or any other material wearable by a patient during an MRI procedure.

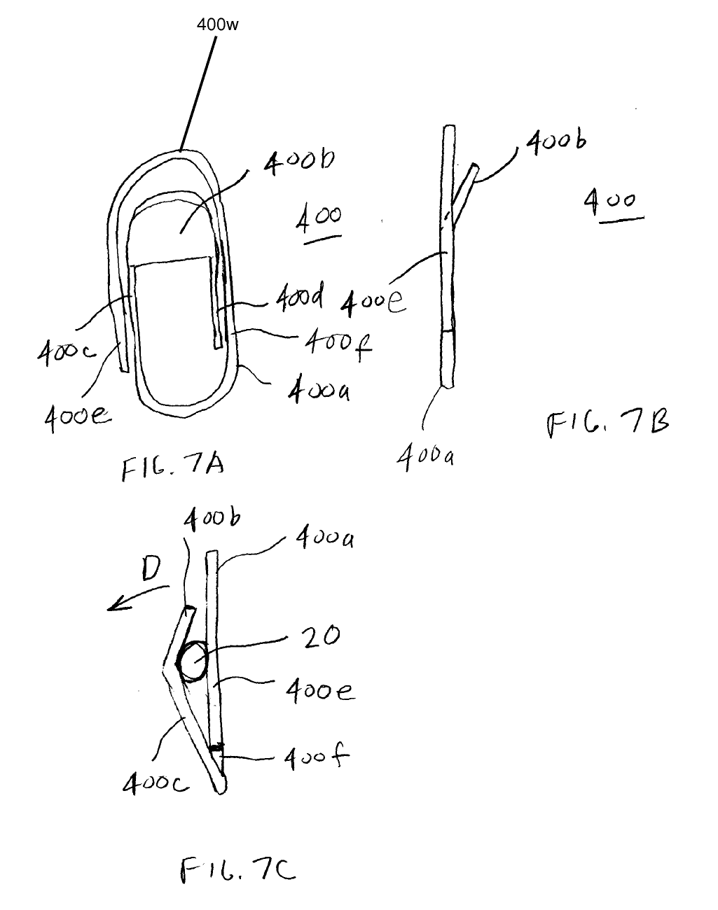

Referring to FIGS. 7A-7C, in another embodiment, the clip 400 has a planar, general spiraling, resiliently deflectable body portion 400a and a tab 400b attached to the body portion and projecting out of a plane defined by the body portion as shown in FIG. 7B. A first leg 400c attaches tab 400b to body portion 400a along a first edge of the tab, and a second leg 400d projects from tab 400b along a second edge of the tab opposite the first edge. Tab 400b is spaced apart from a part 400w of body portion 400a residing opposite the tab a distance sufficient to permit a portion of suspension line 14 to extend between the body portion and tab 400b, so that the clip 400 can be supported by the suspension line in the manner previously described. Body portion 400a also includes a third leg 400e spaced apart from first leg 400c, and a fourth leg 400f spaced apart from second leg 400d. An IV line 20 is inserted between the first and second legs 400c, 400d on one side of the IV and third and fourth legs 400e, 400f on an opposite side of the IV by deflecting tab 400b out of the plane of the body portion, in the direction indicated by arrow D in FIG. 7C, and inserting the IV line between the groups of legs. When the tab is released, the opposed groups of legs attempt to return to their undeflected states, thereby gripping the IV line in an interference fit. The sloped surface of tab 400b aids in preventing movement of the IV line from between the opposed arms. In a particular embodiment, clip 400 is molded or otherwise formed from a polymer material, other non-metallic material or any other material wearable by a patient during an MRI procedure.

In general, the portions of the support mechanism 12 or clips 299 and 400 engaging the IV line are structured to prevent or impede relative movement between the support mechanism 12 or clips 299 and 400 and the IV line in a direction along the line, when the IV line is engaged by the support mechanism 12 or clips 299 and 400. The support mechanism 12 or clips 299 and 400 is also structured to prevent the IV line from inadvertently disengaging from the support mechanism 12 or clips 299 and 400. That is, effort by a user should be required to manipulate one or more portions the support mechanism 12 or clip 299 to disengage the IV line from the support mechanism 12 or clips 299 and 400. Also, the support mechanism 12 or clips 299 and 400 is supportable by suspension line 14.

In one embodiment, the suspension line 14 is in the form of a chain formed of polymeric links suitably connected to each other. However, the suspension line may have any alternative configuration suitable for the purposes described herein. Suspension line 14 may be formed from any suitable material. In a particular embodiment, all of the suspension line components are formed from a polymer material, another non-metallic material or any other material wearable by a patient during an MRI procedure.

The length adjustment mechanism 16 is configured to be releasable by a patient so as to enable a portion or portions of the suspension line 14 to be fed therethrough. As seen in FIGS. 1, 3, and 4, one or more ends of the suspension line are fed through the length adjustment mechanism until a loop 18 (or loops) of a desired size is provided. The loop 18 may be sized so as to fit over and/or around a body part of the patient 22. In one embodiment, loop 18 is sized to fit comfortably around the neck of a patient. However, the loop may be configured fit around any desired portion of the user's body.

The length adjustment mechanism 16 is configured to adjustably grip and retain spaced-apart portions of the suspension line 14, thereby enabling the size of the loop 18 to be adjusted according to the requirements of a particular patient or application.

The length adjustment mechanism 16 is also configured to be lockable by the patient so that a desired position of the adjustment mechanism along the suspension line 14 may be maintained when it is reached, thereby providing and securing a loop 18 of a desired size. The adjustment mechanism 16 may be locked by the patient releasing the mechanism (for example, using a spring-loaded mechanism (as shown)), or the device may be locked by a patient actuating a positive locking feature (not shown) incorporated into the device. The adjustment mechanism 16 may be formed from any suitable material. In a particular embodiment, adjustment mechanism 16 is formed from a polymer material, other non-metallic material or any other material wearable by a patient during an MRI procedure.

Referring to FIG. 3, the IV line 20 is secured to the support mechanism at a location between a first end 20a of the IV line (which is connected to a fluid source, drainage bag, or other medical device 24) and a second end 20b of the IV line (which is inserted into a patient through an incision 199 in the patient's skin). This securement divides the IV line into a first IV line portion 20c extending between the IV line first end and the support mechanism, and a second IV line portion 20d extending between the support mechanism and the insertion site. The IV line support mechanism grips and retains a portion of the IV line 20 spaced a predetermined distance D along the IV line from the insertion site. The support mechanism grips the IV line 20 so as to minimize or eliminate constriction of the IV line 20, and such that a desired flow rate of fluid through the IV line is maintained.

The material(s) and/or structure of the IV line support mechanism 12 are designed to prevent slippage or sliding of the retained portion of the IV line with respect to the support mechanism, when the IV line is secured in the support mechanism. This prevents the second portion 20d of the IV line between the support mechanism and the insertion site from being pulled through the support mechanism. Thus, the length of IV line 20 between the catheter insertion site and the support mechanism remains constant. In this manner, forces on the IV line (for example, the weight of the IV line) that would otherwise tend to extract the catheter from the patient are absorbed by the support mechanism and suspension line. Thus, the support mechanism acts to isolate the second IV line portion 20d from forces acting on the first IV line portion 20c. In particular embodiments, all of the IV line support mechanism components are formed from a polymer material, another non-metallic material or any other material wearable by a patient during an MRI procedure.

In a particular embodiment, each of the suspension line 14, IV support mechanism and the adjustment mechanism 16 are formed exclusively from one or more polymer or other non-metallic materials, thereby enabling the IV support device to be worn under any of a variety of conditions (for example, during an MRI scan). In addition, use of the length adjustment mechanism 16 may permit the size of the loop 18 and/or the configuration of the loop to be adjusted so that IV bags or drainage bags 24 may be supported by the loop.

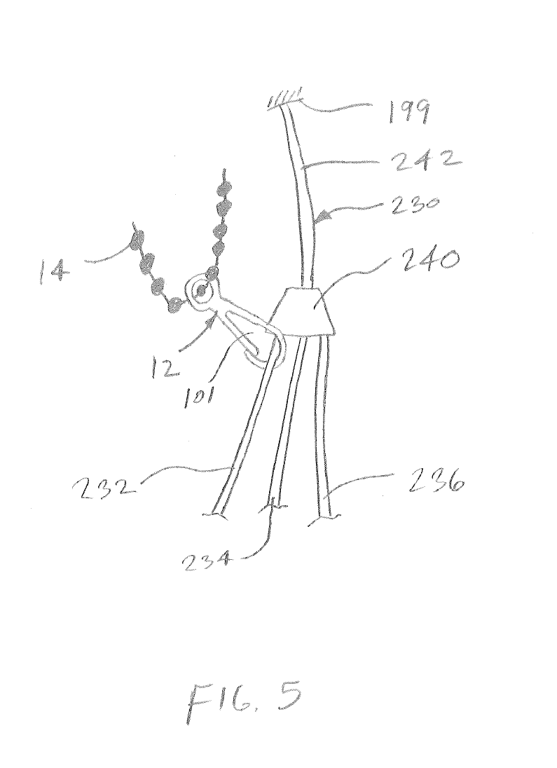

For example, in the embodiment shown in FIGS. 1, 4A and 4B, length adjustment mechanism 16 includes a hollow body portion 16a and a spring-loaded plunger 16b positioned within the body portion. Body portion 16a also has a pair of openings 16d, 16e extending therethrough. Plunger 16b is actuatable by a button 16c located on a side of the body portion 16a and is positioned within the body portion to variably occlude or obstruct openings 16d and 16e.

The openings may be constricted or expanded by operation of button 16c by a user, to move plunger 16b. As seen in FIG. 4A, when button 16c is released, plunger 16b moves in the direction indicated by arrow W to abut portions of suspension line 14 and/or to otherwise constrict the sizes of openings 16d, 16e so as to prevent suspension line 14 from being passed through or fed through the openings 16d, 16e. Conversely, when button 16c is depressed in direction “Y” as shown in FIG. 4B, plunger 16b is depressed, removing a greater portion (or all) of the plunger from the openings 16d, 16d, thereby increasing the opening size to the point where suspension line 14 can be passed through or fed through the openings 16d, 16e. In this manner, button 16c may be depressed and portions of the suspension line passed through the body portion 16a until the desired size of loop 18 is achieved. The button 16c is then released to prevent further passage of the suspension line through the body portion. Thus, the portions of the support line 14 may be fed through the openings 16d, 16e to adjust the sizes of the generated loop 18 to achieve the desired positioning of the IV line support mechanism 12 and any attached IV bag with respect to the patient.

Other configurations of length adjustment mechanism providing the above-described functionality and capabilities are also contemplated.

In a particular embodiment, the components and structure of the support mechanism are specified such that a patent wearing the support mechanism may undergo an MRI (magnetic resonance imaging) procedure, an X-ray procedure, or other similar procedure without the need to remove the support mechanism.

In another particular embodiment, multiple length adjustment mechanisms are used to provide loops of the desired sizes.

It will be understood that the foregoing description of an embodiment of the present invention is for illustrative purposes only. As such, the features herein disclosed are susceptible to a number of modifications commensurate with the abilities of one of ordinary skill in the art, none of which departs from the scope of the present invention as defined in the appended claims.

1. A support device for an intravenous line comprising:
an intravenous line support mechanism;
a suspension line structured for suspending the support mechanism on a portion of a patient's body; and
a suspension line length adjustment mechanism structured to receive portions of the suspension line therethrough, the length adjustment mechanism being structured to enable control of passage of the suspension line therethrough so as to form an adjustable first loop,
the support mechanism including a retaining portion (12b) including a base portion and a resiliently deflectable portion coupled to the base portion to define a second loop (301) therebetween, the second loop (301) being structured for receiving and securing a portion of at least one intravenous line therein, wherein the deflectable portion is structured to be deflected in a direction away from the second loop to enable insertion of the at least one intravenous line into the second loop.

2. The support device of claim 1, wherein the support mechanism includes a line receiving portion (12a) for attachment to the suspension line thereto, and wherein the retaining portion is structured for securing the support mechanism to an intravenous line at a desired location along the intravenous line.

3. (canceled)

4. The support device of claim 1 wherein the deflectable portion has a hooked end structured to prevent inadvertent extraction of the at least one intravenous line from the second loop.

5. The support device of claim 1 wherein the line support mechanism is structured to maintain a predetermined portion of an intravenous line in contact therewith.

6. The support device of claim 5 wherein the base portion and the resiliently deflectable portion are structured to engage an intravenous line extending through the second loop in an interference fit between the base portion and the resiliently deflectable portion.

7. The support device of claim 1, wherein the length adjustment mechanism includes a housing having a plurality of passages extending therethrough, each passage being structured for receiving a portion of the suspension line therein.

8. The support device of claim 7 wherein the housing has at least one opening structured for receiving a portion of a user-actuatable button therein.

9. The support device of claim 8 wherein the button is coupled to a plunger so as to enable constriction and expansion of the plurality of passages through movement of the button by a user.

10. The support device of claim 1 wherein the intravenous line support mechanism, suspension line, and suspension line length adjustment mechanism are all formed from polymeric materials.

11. A support device for an intravenous line comprising:
an intravenous line support mechanism;
a suspension line structured for suspending the support mechanism on a portion of a patient's body; and
a suspension line length adjustment mechanism structured to receive portions of the suspension line therethrough, the length adjustment mechanism being structured to enable control of passage of the suspension line therethrough so as to form an adjustable first loop,
the support mechanism including a base portion (299c) and an arm (299d) extending from the base portion (299c) and opposite the base portion, the arm being resiliently deflectable away from the base portion, the base portion and the arm combining to define a second loop (301) structured for receiving at least a portion of an intravenous line therein, the arm (299d) having a hooked portion (299e) extending from an end of the arm in a direction toward the second loop (301).

12. The support device of claim 11 wherein a straight portion extends from the hooked portion (299e) and is angled with respect to the base portion (299c) so as to provide a lead-in surface leading into the loop (301).

13. A support device for an intravenous line comprising:
an intravenous line support mechanism;
a suspension line structured for suspending the support mechanism on a portion of a patient's body; and
a suspension line length adjustment mechanism structured to receive portions of the suspension line therethrough, the length adjustment mechanism being structured to enable control of passage of the suspension line therethrough so as to form an adjustable loop,
the support mechanism including a planar, spiraling, resiliently deflectable body portion (400a), and a movable tab (400b) attached to the body portion (400a) and projecting out of a plane defined by the body portion (400a),
wherein a first portion of the body portion is structured to resiliently deflect with respect to a second portion of the body portion and out of a plane of the body portion, so as to receive an intravenous line between the first and second portions of the body portion.

14. The support device of claim 13 wherein a first leg (400c) attaches tab (400b) to body portion (400a) along a first edge of the tab, and a second leg (400d) projects from tab (400b) along a second edge of the tab opposite the first edge.

15. The support device of claim 1 wherein the loop (101) is sized for receiving a plurality of intravenous lines therein.

16. The support device of claim 11 wherein the hooked portion defines a portion of the second loop.

17. The support device of claim 11 wherein the hooked portion is structured to prevent inadvertent extraction of an IV line from second loop in a direction opposite a direction of insertion of the IV line into the second loop,_by contacting a portion of the IV line.

18. The support device of claim 13 wherein the support mechanism is structured so that an intravenous line is securable between the first portion of the body portion, the second portion of the body portion, and the tab when the first portion of the body portion is deflected out of a plane of the body portion with respect to a second portion of the body portion.

19. The support device of claim 13 wherein the tab is attached to the first portion of the body portion so as to move with the first portion of the body portion when the first portion of the body portion is resiliently deflected.
